(19) United States
(12) Patent Application Publication
(10) Pub. No.: US 2026/0000502 A1
(43) Pub. Date: Jan. 1, 2026

(54) SURGICAL IMPLANT DELIVERY DEVICE

(71) Applicant: Song Hee KIM, Anyang-si (KR)

(72) Inventor: Song Hee KIM, Anyang-si (KR)

(21) Appl. No.: 18/781,281

(22) Filed: Jul. 23, 2024

(30) Foreign Application Priority Data
Jun. 28, 2024 (KR) 10-2024-0085476

(51) Int. Cl. A61F 2/12

(52) U.S. Cl. CPC A61F 2/12

(57) ABSTRACT

A surgical implant delivery device is provided. The surgical implant delivery device comprises a sleeve. The sleeve includes a delivery portion and an accommodating portion. The delivery portion has an opening. The accommodating portion extending from the delivery portion. The delivery portion includes a wing portion protruding widthwise. The wing portion is foldable to reduce the width of the opening.

### CROSS-REFERENCE TO RELATED APPLICATIONS

This application claims benefit of priority to Korean Patent Application No. 10-2024-0085476 filed Jun. 28, 2024, the entire content of which is incorporated herein by reference.

### TECHNICAL FIELD

The present disclosure relates to a surgical implant delivery device, and more particularly, to a surgical implant delivery device including a sleeve.

### BACKGROUND

The contents described in this part simply provide background information on the present disclosure and do not constitute prior art.

While it has been mentioned herein that the device may be utilized to deliver pre-filled breast implants in particular, the surgical implants that may be delivered by the device of the present invention are not limited to pre-filled breast implants.

Surgical techniques are continuously advancing, with ongoing efforts to reduce incision size and the potential for infection, thereby minimizing trauma to the body from surgery, reducing postoperative scarring, and decreasing the need for secondary surgeries. This is particularly significant in the field of breast augmentation and reconstruction, where the long-term appearance of the breast is an important consideration in determining the success of the surgery.

Many patients prefer pre-filled breast implants (e.g., silicone implants) because pre-filled implants can provide a more natural appearance compared to on-site filled implants. However, pre-filled implants are inherently larger, requiring larger incisions. This necessitates the development of delivery devices that can place pre-filled breast implants into the surgical pocket through smaller incisions without damaging the implants. One such solution is the Keller Funnel® developed and sold by Keller Medical Inc., as described in U.S. Pat. No. 8,211,173. The Keller Funnel® is a piping bag-like sleeve, wherein the breast implant is inserted into the larger end of the sleeve and then pushed through the smaller end of the sleeve into the patient's incision pocket.

However, this model of implant delivery device has a problem in that, during the process of compressing the sleeve to expel the implant, unintended leakage of air inside the sleeve through the larger end can occur, leading to a loss of internal pressure needed to expel the implant from the sleeve.

### SUMMARY

Therefore, the present disclosure has been made to solve the above-mentioned problems, and an object thereof is to provide a surgical implant delivery device that can prevent the loss of internal pressure within the sleeve during the compression process.

The problems to be solved by the present disclosure are not limited to the problems mentioned above, and other problems not mentioned will be clearly understood by those skilled in the art from the description below.

According to one embodiment of the present disclosure, there is provided a surgical implant delivery device comprising: a sleeve, wherein the sleeve includes a delivery portion having an opening, and an accommodating portion extending from the delivery portion to the opposite side of the opening and communicating with the delivery portion, wherein the delivery portion includes a wing portion protruding widthwise, and wherein the wing portion is foldable to reduce the width of the opening.

The surgical implant delivery device may further comprise a fastener for securing the wing portion in a folded state.

The fastener may comprise a velcro tape.

The wing portion may include a first wing and a second wing protruding in opposite directions when unfolded, and the fastener may include a first velcro attached to the first wing and a second velcro attached to the second wing.

The fastener may have protruding tab protruding outward from the sleeve.

The first wing and the second wing may include outwardly sloping first inclined portion and inwardly sloping second inclined portion, and the first velcro may comprise a body portion attached along an outer periphery of the first inclined portion and the second inclined portion, and a velcro tab protruding outward from the body portion.

The delivery portion may have a single-layer portion.

The accommodating portion may comprise an air outlet formed at an end opposite to the opening.

The sleeve may comprise a beveled portion connecting the delivery portion with the accommodating portion slantly.

The accommodating portion may have a first edge and a second edge that are parallel to each other or converge toward the opening, and the wing portion may protrude further in the width direction than points where an extension of the first edge and an extension of the second edge meet a distal end of the sleeve.

The sleeve may have printed guide line corresponding to an extension of the first edge and an extension of the second edge.

According to the present invention, there is provided a surgical implant delivery device capable of preventing loss of internal pressure of the sleeve while external force is applied to the sleeve.

### BRIEF DESCRIPTION OF THE DRAWINGS

FIG. 1 is a front view of a surgical implant delivery device according to one embodiment of the present invention in a unfolded state.

FIG. 2 is a rear view of a surgical implant delivery device according to one embodiment of the present invention in an unfolded state.

FIG. 3 is a front view of a surgical implant delivery device according to one embodiment of the present invention in a folded state.

FIG. 4 is a rear view of a surgical implant delivery device according to one embodiment of the present invention in a folded state.

FIG. 5 illustrates a surgical implant delivery device in use according to one embodiment of the present invention.

### DETAILED DESCRIPTION

Advantages and features of the present disclosure, and methods of achieving them, will become clear with reference to the detailed description of the following embodiments taken in conjunction with the accompanying drawings. However, the present disclosure is not limited to the embodiments disclosed below, but will be implemented in various different forms, these embodiments are provided to make the disclosure of the present disclosure complete, and merely provided to completely inform those skilled in the art to which the present disclosure belongs of the scope of the invention, and the present disclosure is only defined by the scope of the claims. Thus, in some embodiments, well-known process steps, well-known device structures, and well-known techniques have not been described in detail in order to avoid obscuring the interpretation of the present disclosure. Like reference numbers designate like elements throughout the specification.

In the drawings, the thickness is illustrated enlarged to clearly express the various layers and regions. Like reference numerals have been assigned to like parts throughout the specification. When a part such as a layer, film, region, plate, or the like is said to be “on” another part, this includes not only the case where it is “directly on” the other part, but also the case where there is another part therebetween. Conversely, when a part is said to be “directly on” another part, it means that there is no other part therebetween. In addition, when a part such as a layer, film, region, plate, or the like is said to be “below” another part, this includes not only the case where it is “directly below” the other part, but also the case where there is another part therebetween. Conversely, when a part is said to be “directly below” another part, it means that there is no other part therebetween.

The spatially relative terms such as “below”, “beneath”, “lower”, “above”, “upper”, or the like may be used to readily describe the relationship between one element or element and another element or element as illustrated in the drawings. The spatially relative terms should be understood as encompassing different orientations of elements in use or operation in addition to the orientations illustrated in the drawings. For example, when elements illustrated in the drawings are reversed, elements described as “below” or “beneath” other elements may be placed “above” the other elements. Thus, the exemplary term “below” may include directions of both below and above. Elements may also be oriented in other orientations, and thus, the spatially relative terms may be interpreted according to orientation.

In this specification, when a part is said to be connected to another part, this includes not only the case where it is directly connected, but also the case where it is connected with another element interposed therebetween. In addition, when a part includes a certain component, it means that it may further include other components without excluding other components unless otherwise specified.

In this specification, terms such as first, second, and third may be used to describe various components, but these components are not limited by the terms. The terms are used for the purpose of distinguishing one component from other components. For example, a first component could be termed a second or third component, or the like, and similarly, a second or third component could also be termed interchangeably, without departing from the scope of the present disclosure.

Unless otherwise defined, all terms (including technical and scientific terms) used in the present specification may be used in a meaning commonly understood by those of ordinary skill in the art to which the present disclosure belongs. In addition, terms defined in commonly used dictionaries are not interpreted ideally or excessively unless explicitly specifically defined.

A surgical implant delivery device of the present invention comprises a sleeve. The sleeve includes a delivery portion and an accommodating portion. The delivery portion has an opening. The accommodating portion extending from the delivery portion to the opposite side of the opening and communicating with the delivery portion. The delivery portion includes a wing portion protruding widthwise. The wing portion is foldable to reduce the width of the opening.

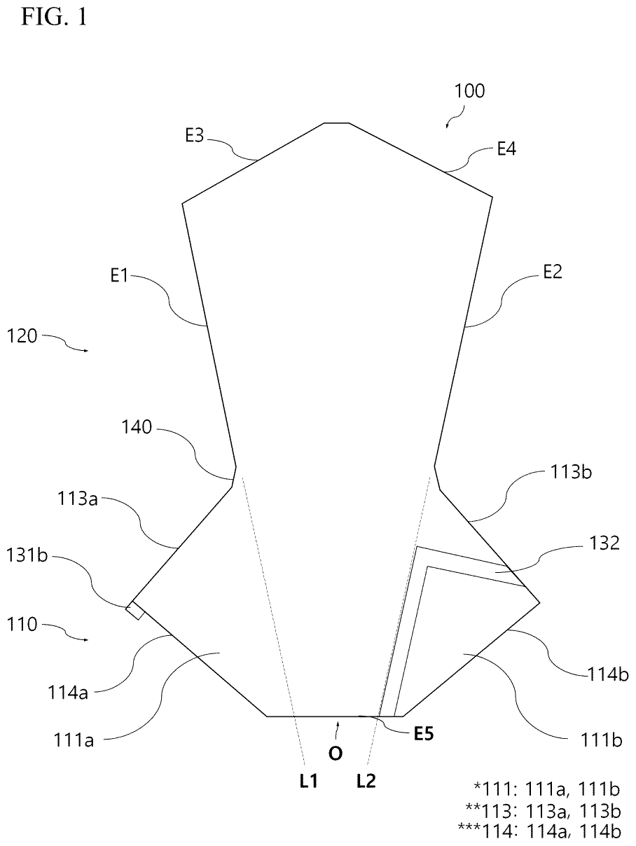

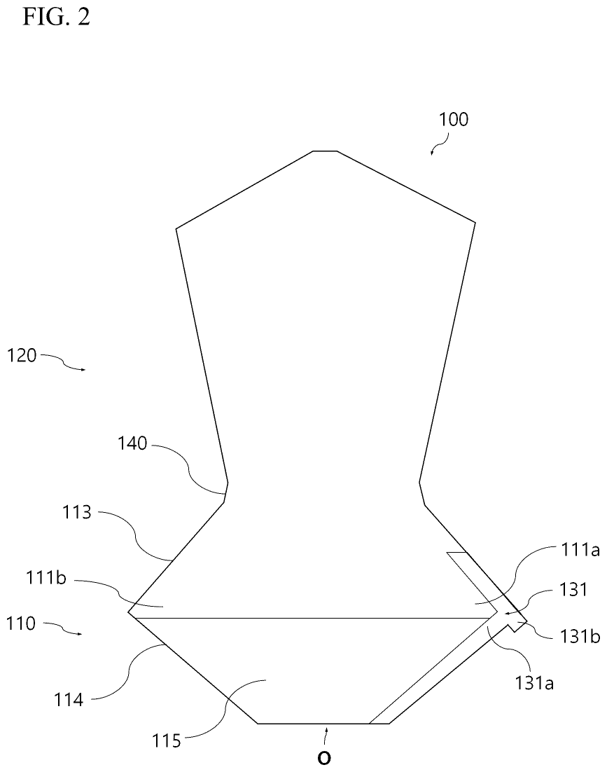

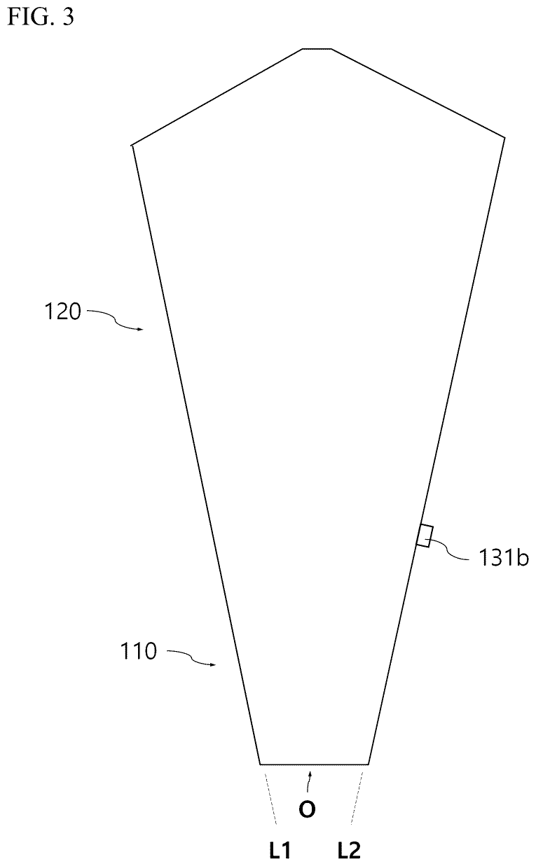

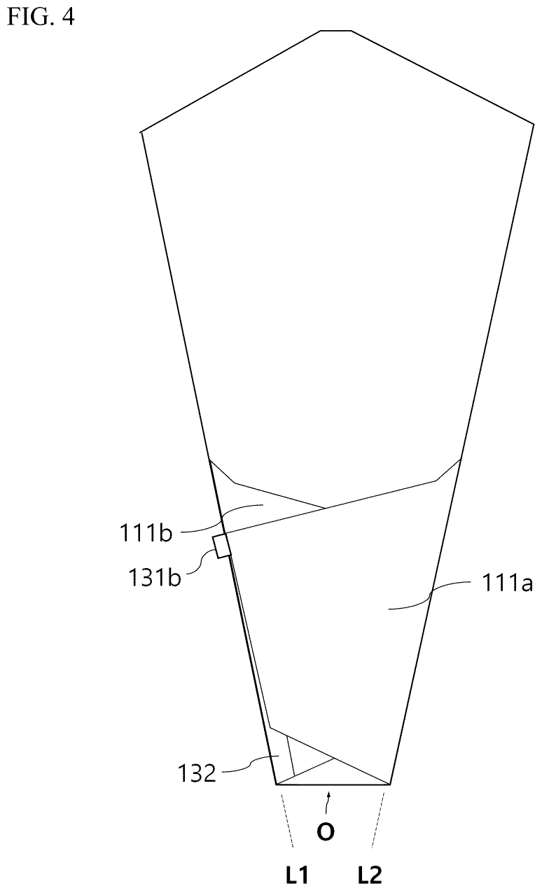

Hereinafter, a surgical implant delivery device according to one embodiment of the present disclosure will be described with reference to FIGS. 1 to 5. FIG. 1 is a front view of a surgical implant delivery device according to one embodiment of the present invention in an unfolded state. FIG. 2 is a rear view of a surgical implant delivery device according to one embodiment of the present invention in an unfolded state. FIG. 3 is a front view of a surgical implant delivery device according to one embodiment of the present invention in a folded state. FIG. 4 is a rear view of a surgical implant delivery device according to one embodiment of the present invention in a folded state. In Figures, the sleeve 100 of the surgical implant delivery device is shown as opaque for ease of illustration, but the surgical implant delivery device of the present invention may be transparent.

Referring to FIGS. 1 to 4, a surgical implant delivery device according to one embodiment of the present invention comprises sleeve 100, wherein the sleeve 100 includes delivery portion 110, accommodating portion 120, fastner 131,132 and beveled portion 140.

The delivery portion 110 has an opening O. The accommodating portion 120 extending from the delivery portion 110 to the opposite side of the opening O and communicationg with the delivery portion 110. The delivery portion 110 includes a wing portion 111 protruding widthwise. The wing portion 111 is foldable to reduce the width of the opening O. The sleeve 100 may have a shape symmetrical to the longitudinal axis. An opening O of the delivery portion 110 may be formed at a distal end of the sleeve 100.

The accommodating portion 120 may have a first edge E1 and a second edge E2 that are parallel to each other or converge toward the opening O. In one embodiment, an extension L1 of the first edge E1 and an extension L2 of the second edge E2 form an acute angle with each other. The first edge E1 and the second edge E2 may define an implant receiving space that tapers in cross-section towards the opening O. The accommodating portion 120 may have third edge E3 and fourth edge E4 extending from the first and second edges E1, E2 to the opposite side of the opening O. The edges E1, E2, E3, E4 of the accommodating portion 120 may form obtuse angles with the adjacent edges. However, the shape of the accommodating portion 120 is not limited to the embodiment, and for example, the outline of the accommodating portion 120 may follow a curved path at least in part. In other embodiments, the accommodating portion 120 may include an air outlet formed at an end opposite to the opening (i.e., the proximal end). With the sleeve 100 unfolded, the width of the distal end of the accommodating portion 120 may be narrower than the width of the opening O.

In one embodiment of the invention, a surgical implant 1 may be inserted into the sleeve 100 through the opening O of the delivery portion 110, and then may be ejected from the sleeve 100 through the opening O to the outside of the sleeve 100. The accommodating portion 120 of the surgical implant delivery device according to one embodiment of the present invention is externally communicated only through the opening O of the delivery portion 110, i.e., the accommodating portion 120 has no other openings other than the communicating pathway to the delivery portion 110. This prevents inadvertent loss of internal pressure in the sleeve 100 when the sleeve 100 is pressurized to expel the implant 1 from the sleeve 100.

The wing portion 111 may include a first wing 111a and a second wing 111b protruding in opposite directions when unfolded. The wing portion 111 may project outwardly beyond an extension L1 of the first edge E1 and an extension L2 of the second edge E2 of the accommodating portion 120. The wing portion 111 may include outwardly sloping first inclined portion 113 and inwardly sloping second inclined portion 114. In one embodiment, each of the first wing 111a and second wing 111b includes an outwardly sloping first inclined portion 113 and an inwardly sloping second inclined portion 114 extending from the first inclined portion 113 to the opening O. The angle formed by an extension of the second inclined portion 114a of the first wing 111a and an extension of the second inclined portion 114b of the second wing 111b may be greater than the angle formed by an extension L1 of the first edge E1 of the receptacle 120 and an extension L2 of the second edge E2 of the accommodating portion 120. However, the wing portion 111 of the present invention is not limited to this embodiment, and the wing portion 111 may, for example, have only one protruding wing. The width of the opening O when the wing portion 111 is unfolded is larger than the width of the opening O when the wing portion 111 is folded.

The sleeve 100 may have a front layer and a rear layer overlapping at least a portion of the front layer. The delivery portion 110 may have a single-layer portion 115. In one embodiment of the present invention, the delivery portion 110 has a single-layer portion 115 such that the distal end of the front layer of the delivery portion 110 is not overlapped, and the inner surface of the front layer is exposed to the rear when the wing portion 111 is unfolded. The single-layer portion 115 may occupy approximately one-half of the length of the delivery portion 110 (in the direction connecting the distal and proximal ends of the sleeve). The single-layer portion 115 may form at least a portion of the second inclined portion 114. In devices that do not have the single-layer portion, when the wing portion is folded, there is an overlap of the four layers of the sleeve near the opening. The inventors of the present invention have found that this overlap of 4 layers can obstruct expansion of the sleeve opening and can eventually cause unintentional tearing of the opening. In the present invention with the single-layer portion 115, only one or two layers are stacked near the opening O when the wing portion 111 is folded. Thus, the present invention with the single-layer portion 115 does not hinder the expansion of the opening O and can prevent the opening O from tearing.

The beveled portion 140 connects the delivery portion 110 with the accommodating portion 120 slantly. The inventor of the present invention observed that without the beveled portion 140, the portion between the delivery portion 110 and the accommodating portion 120 can be burst by the pressure applied to the sleeve 100. The beveled portion 140 prevents such damage.

The distal end of the delivery portion 110 may have a widthwise end edge E5. With the sleeve 100 unfolded, an extension L1 of the first edge E1 and an extension L2 of the second edge E2 may pass the end edge E5. The wing portion 111 may protrude further in the width direction than points where an extension L1 of the first edge E1 and an extension L2 of the second edge E2 meet a distal end of the sleeve 100. The sleeve 100 may have printed guide line corresponding to an extension L1 of the first edge E1 and an extension L2 of the second edge E2. Referring to FIGS. 3 and 4, with the wing portion 111 folded along the guide line, the folded edges of the delivery portion 110 are parallel to the first and second edges E1, E2 of the sleeve 100.

The fastener 131, 132 secures the wing portion 111 in a folded state. The fastener 131,132 may comprise a velcro tape 131,132 or may be the velcro tape 131,132 itself. The fastener 131,132 may include a first velcro 131 attached to the first wing 111a and a second Velcro 132 attached to the second wing 111b. The first Velcro 131 may be attached to the rear layer of the sleeve 100 and the second Velcro 132 may be attached to the front layer of the sleeve 100. The fastener 131,132 may have a velcro tab 131b that protrude outward from the sleeve 100. The first velcro 131 may be a hook velcro and the second velcro 132 may be a ring velcro. However, the configuration of the fastener 131,132 is not limited to this embodiment. The fastener 131,132 may comprise, for example, buttons. The second velcro 132 may have a fiber layer and an adhesive layer, and a support layer may be attached between the second Velcro 132 and the sleeve 100 to prevent the fiber layer of the ring velcro from being frayed by frequent adhesion and detachment with the hook velcro. The support layer may comprise polylactic acid (PLA).

In one embodiment, the first Velcro 131 comprises a body portion 131a attached along an outer periphery of the first inclined 113 portion and the second inclined portion 114, and a velcro tab 131b protruding outward from the body portion 131a.

The second velcro 132 may have a shape corresponding to the body portion 131a of the first velcro 131. At least a portion of the second velcro 132 may be attached abuttingly to an extension L2 of the second edge E2.

At least a portion of the sleeve 100 may be made of a transparent, flexible synthetic resin. The sleeve 100 may have printed instructions to guide where to incise the delivery portion 110 based on the folding sequence of the first wing 111a and second wing 111b and the size of the implant.

The surgical implant delivery device of the present invention may include an opening tab (not shown) that assists in opening the sleeve 100. The opening tab may have an attachment portion that attaches to the delivery portion 110 and a free end portion extending from the attachment portion. The free end may be printed with instructions to guide the pulling direction. The opening tab may be attached to the rear layer of the sleeve 100. The opening tab may be provided in the form of a film. The opening tab may be a film.

The surgical implant delivery device of the present invention may include a sanitary paper (not shown) with a portion inserted into the interior of the sleeve 100 and another portion protruding from the sleeve 100. The sanitary paper may have a shape corresponding to the shape of the front layer of the sleeve 100. The sanitary paper prevents the front layer and rear layer of the sleeve 100 from adhering to each other.

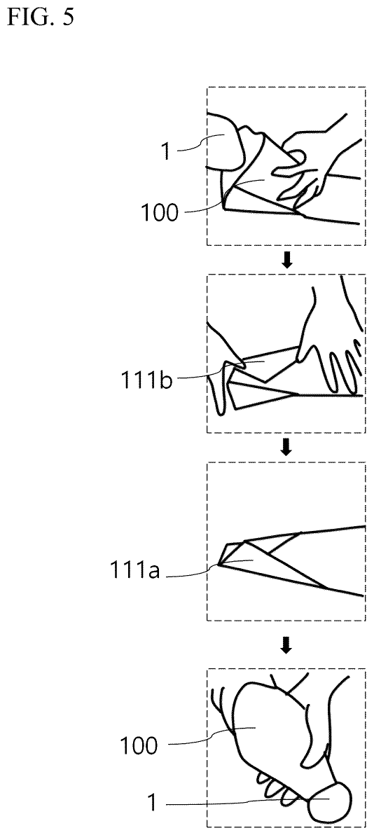

FIG. 5 illustrates a surgical implant delivery device in use according to one embodiment of the present invention. Referring to FIG. 5, a surgeon opens the opening O with the wings 111 unfolded and inserts the surgical implant 1 into the sleeve 100 through the opening O. The surgeon then folds the second wing 111b to the rear side of the sleeve 100. The surgeon then folds the first wing 111a to the rear side of the sleeve 100. At this point, the first velcro 131 and the second velcro 132 are attached to each other to secure the wing 111 in the folded position. Then, when the surgeon applies external pressure to the accommodating portion 120, the implant 1 is ejected from the sleeve 100 through the opening O and can be delivered to the patient's body.

The above description is merely an example of the technical idea of the present disclosure, and those skilled in the art can make various modifications, changes, and substitutions without departing from the essential characteristics of the present disclosure. Therefore, the embodiments are not intended to limit the technical idea of the present disclosure, but to explain, and the scope of the technical idea of the present disclosure is not limited by these embodiments. The protection scope of the present disclosure should be construed according to the claims below, and all technical ideas within the equivalent range should be construed as being included in the scope of the present disclosure.

[Detailed Description of Main Elements]

| | |
|---|---|
| 100: sleeve | 110: delivery portion |
| 111: wing portion | 111a: first wing |
| 111b: second wing | 113: first inclined portion |
| 114: second inclined portion | 115: single-layer portion |
| 120: accommodating portion | 130: fastener |
| 131: first velcro | 131a: body portion |
| 131b: velcro tab | 132: second velcro |

What is claimed is:

1. A surgical implant delivery device comprising:
a sleeve,
wherein the sleeve includes a delivery portion having an opening, and an accommodating portion extending from the delivery portion to the opposite side of the opening and communicating with the delivery portion,
wherein the delivery portion includes a wing portion protruding widthwise, and
wherein the wing portion is foldable to reduce the width of the opening.

2. The surgical implant delivery device of claim 1, further comprising a fastener for securing the wing portion in a folded state.

3. The surgical implant delivery device of claim 2, wherein the fastener comprises a velcro tape.

4. The surgical implant delivery device of claim 3, wherein the wing portion includes a first wing and a second wing protruding in opposite directions when unfolded, and
the fastener includes a first velcro attached to the first wing and a second velcro attached to the second wing.

5. The surgical implant delivery device of claim 3, wherein the fastener has protruding tab protruding outward from the sleeve.

6. The surgical implant delivery device of claim 4, wherein the first wing and the second wing include outwardly sloping first inclined portion and inwardly sloping second inclined portion, and
the first velcro comprises a body portion attached along an outer periphery of the first inclined portion and the second inclined portion, and a velcro tab protruding outward from the body portion.

7. The surgical implant delivery device of claim 1, wherein the delivery portion has a single-layer portion.

8. The surgical implant delivery device of claim 1, wherein the accommodating portion comprises an air outlet formed at an end opposite to the opening.

9. The surgical implant delivery device of claim 1, wherein the sleeve comprises a beveled portion connecting the delivery portion with the accommodating portion slantly.

10. The surgical implant delivery device of claim 1, wherein the accommodating portion has a first edge and a second edge that are parallel to each other or converge toward the opening, and
the wing portion protrudes further in the width direction than points where an extension of the first edge and an extension of the second edge meet a distal end of the sleeve.

11. The surgical implant delivery device of claim 10, wherein the sleeve has printed guide line corresponding to an extension of the first edge and an extension of the second edge.
